(19) United States
(12) Patent Application Publication
(10) Pub. No.: US 2012/0171930 A1
(43) Pub. Date: Jul. 5, 2012

(54) Bra Having Therapeutically Positioned Pockets as well as Hot and or Cold Inserts

(76) Inventor: Allison Kaufman, Miami, FL (US)

(21) Appl. No.: 12/983,930

(22) Filed: Jan. 4, 2011

(51) Int. Cl. A41C 3/02; A61F 7/00; A41C 3/12

(52) U.S. Cl. 450/58; 450/89; 450/86; 607/112

(57) ABSTRACT

A therapeutic bra having a first cup connected to a first strap and a second cup connected to a second strap where the first strap is further connected to a backing material and the second strap is further connected to the backing material so that a first piece of material forms a first pocket with a portion of the first cup's material in specific physical symmetry with a location of a surgical injury on a first breast of a patient. Similarly, a second piece of material forms a second pocket with a portion of the second cup's material in specific physical symmetry with a location of a surgical injury on a second breast of a patient. The first and second cups' pockets mimic the shapes of injuries sustained on the respective breasts that they support. Cold, hot and or hot-cold gel packs are insert into the pockets for therapeutic purposes.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the United States Patent and Trademark Office patent file or records, but otherwise reserves all copyrights whatsoever.

### BACKGROUND

(1) Field of the Invention

This invention relates to improvements in the field of therapeutic bras used for providing support to women undergoing pain or discomfort as a result of surgery.

(2) Description of the Related Art

US Patent Citations

U.S. Pat. No. 7,275,977 to Rhodes teaches a therapeutic bra (100) for reducing breast tissue inflammation in a woman who has recently undergone breast surgery. The therapeutic bra (100) is made up of a sports bra (110), which comprises a front breast section (120), opposite bra sides (140, 160), first (180) and second (200) shoulder straps, and a rear section (220) located between the first (140) and second (160) opposite bra sides. The front breast section (120) comprises an outer layer (260) made up of hook and loop fastener material (125) such as hooks, loops, alone or in combination, and a therapeutic pouch (115). The therapeutic pouch (115) comprises front (280) and rear (300) sides, an opening (320), and an interior (330) accessible via opening (320). The rear side (300) is at least partly covered with complementary hooks, loops, alone or in combination (310) to reversibly attach to the outer layer (260).

In Particular, Rhodes teaches a therapeutic bra 100 for patients that have undergone breast surgery. The bra 100 has a front section 120 has an outer layer 260 made up of hook and or loop fasteners that are used to attach a therapeutic pouch 115 having a rear side that is covered with complementary hook and or loop fasteners. This side of the pouch is designed to attach to the outer layer 260 of the sports bra. The pouch 115 has a cold pack, a hot pack or a hot-cold pack inserted therein for therapeutic purposes. Of course, an individual who has suffered a stressful surgical procedure sometimes does not carefully monitor his actions after said surgery. If she decides to remove the pouch from the bra a painful snapping motion results that is typical of Velcro type material utilized in Rhodes. This will reverberate across the entire breast area, rib area and upper chest area; indeed there will be pain for the person who rips this pouch without thinking what is going to happen. Therefore, there is a need to overcome these deficiencies found in Rhodes.

U.S. Pat. No. 7,081,034 to Zoellner teaches a nursing bra comprised of two cups with an outer pocket/flap in each cup for holding a thin circular heating/cooling device in place. This pocket is a full piece of fabric with an opening at the upper center portion of the garment that holds the heating/cooling device in place. The heating/cooling device provides relief from engorgement, plugged ducts, mastitis and other general nursing pain. There is also a rear panel comprised of two cups with openings in each cup for nursing a baby. The front and rear panel coexist, forming a supportive and comfortable bra. The removable front, outer panel is connected to the rear panel at the base, which allows for the upper portion of the front panel to be lowered allowing the nipple, and surrounding area of the woman's breast to be exposed for nursing. Zoellner's teaching help reduce the discomfort of a mother from nursing related discomfort but do not teach a mechanism to solve surgery related discomfort. Thus, there needs to be a solution to overcome this deficiency.

U.S. Pat. No. 6,464,717 to Smith et al. teaches a bra with hot/cold inserts is a therapeutic device in the form of a vest-like elastic garment adapted to be worn on the human upper torso. The device includes front panels having pockets therein for retaining gel packs. The device is effective in providing warm or cold therapy to the chest and rib areas. The garment can be used by humans of both genders.

In Particular Smith's vest 10 garment is directed to providing cold or warm therapy to the chest and rib areas using hot or cold gel packs. In so doing the entire area will be affected by the warmth or cold provided by the hot or cold gel packs that are inserted therein; this therapy even affects areas of the breast that have not been operated upon. Thus, there needs to be a solution to the deficiencies found in Smith.

### BRIEF SUMMARY OF THE INVENTION

Herein is described a therapeutic bra comprising a first cup connected to a first strap; a second cup connected to a second strap; a first strap further connected to a backing material; a second strap further connected to the backing material; and a first piece of material forming a first pocket with a portion of the first cup's material in specific physical symmetry with a location of a surgical injury on a first breast of a patient. The therapeutic bra further comprises a second piece of material forming a second pocket with a portion of the second cup's material in specific physical symmetry with a location of a surgical injury on a second breast of a patient. Also, the first piece of material forming a first pocket with a portion of the first cup's material in specific physical symmetry with a location of a surgical injury on a first breast of a patient is situated on the internal side of the first cup. Likewise, the second piece of material forming a second pocket with a portion of the second cup's material in specific physical symmetry with a location of a surgical injury on a second breast of a patient is situated on the internal side of the second cup.

Optionally, the first piece of material forming a first pocket with a portion of the first cup's material in specific physical symmetry with a location of a surgical injury on a first breast of a patient is situated on the external side of the first cup. Likewise, optionally, the second piece of material forming a second pocket with a portion of the second cup's material in specific physical symmetry with a location of a surgical injury on a second breast of a patient is situated on the external side of the second cup. Whether located external to the side of the cup exposed to the air or cushioned in direct contact with the breast the pockets themselves are to be filled with gel packs that preferably mimic the overall shape of the pockets themselves. Alternatively, the gel packs can be of any shape that when combined together and in association with the snug pocket material brings a snug fit to the pockets closure. Another embodiment has only one cup with a pocket.

The first and second cups' pockets are shaped in a variety of fashions to match the injuries sustained during the surgical process. Typical shapes that they can take are half-moons; horizontal kidneys; and more generically, according to the shapes of injuries sustained on the respective breasts that they support. The therapeutic bra is connected via the first and second straps to the backing material through a central material that is connected to the backing material. Alternatively, there is no central material or backing material only a direct connection of each strap independently to the curved material that proceeds from one cup end to the other cup's end. The overall system has at least two columns of vertical fasteners attached each nearby an opposite end of a cup so as to provide front bra closure when engaged one column of vertical fasteners to the other. Optionally, there are more than two columns of vertical fasteners attached nearby ends of the cup supporting material so as to provide front bra closure when engaged one selected column to a selected column at another end. Additionally, the system includes an optional Velcro material of hoops and links on a flap-external lip combination closure or a internal lip and side wall combination closure.

### BRIEF DESCRIPTION OF THE SEVERAL VIEWS OF THE DRAWINGS

FIG. 1 illustrates a front perspective view of a spa bra in accordance with the teachings of the preferred embodiment.

FIG. 2 illustrates a inside perspective view of an open spa bra with the several fasteners detached from one another in accordance with the teachings of the preferred embodiment.

FIG. 3 illustrates the insertion of hot, cold or hot-cold gel packs into a pocket in accordance with the teachings of the preferred embodiment.

FIG. 4 illustrates an optional aspect of an alternative embodiment showing the closure of pockets utilizing small external and internal strips of corresponding hook and or loop strips of material in accordance with the teachings of the embodiment.

### DETAILED DESCRIPTION OF THE INVENTION

Although the disclosure herein is detailed and exact to enable those skilled in the art to practice the invention, the physical embodiments herein disclosed merely exemplify the invention that may be embodied in other specific structures. While the preferred embodiment has been described, the details may be changed without departing from the spirit of the invention that is defined by the claims.

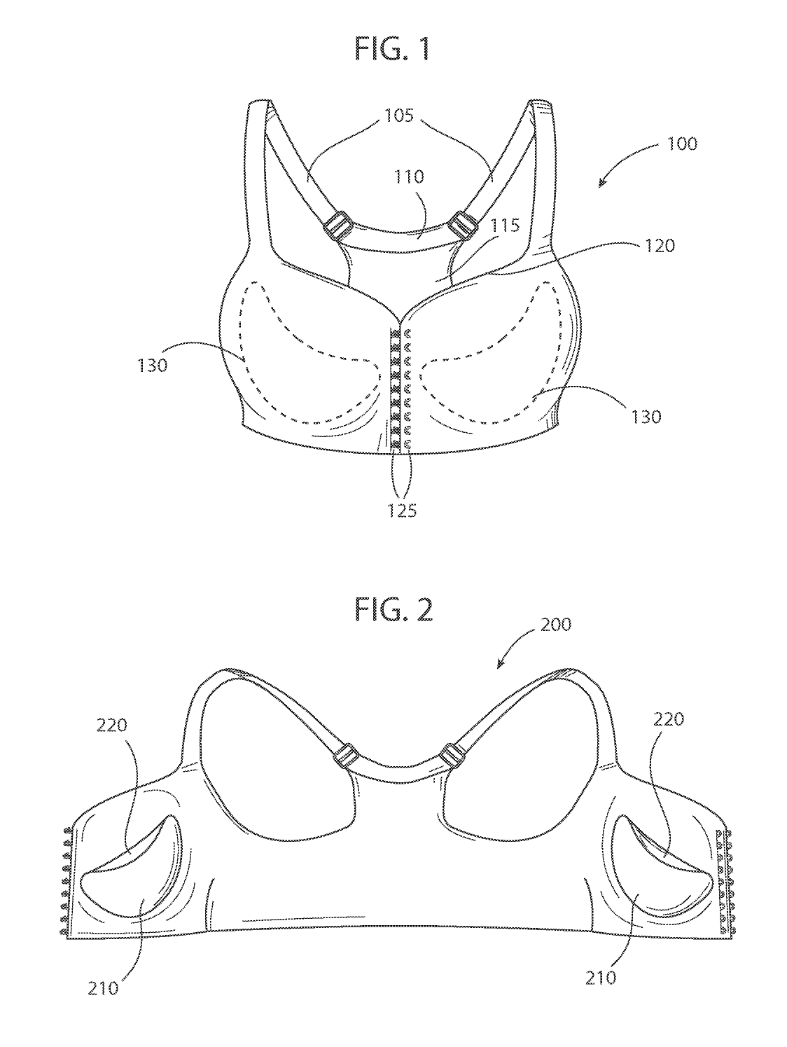

FIG. 1 illustrates a front perspective view of a spa bra in accordance with the teachings of the preferred embodiment. The spa therapeutic bra of the present preferred embodiment is generally indicated as 100. The entire fabric of the bra is preferably made from soft, expandable materials that hold well their new shape and retract to their original form if necessary. There are numerous types of fabric blends that expand outwards yet are capable of holding a new shape well. Some typical blends that conform to this standard include 51% nylon and 49% soft lycra as well as another blend of 75% nylon, 15% polyester and 10% spandex; these blends are exemplary blends and it should be understood that other material choices have the potential to fulfill the needs required herein.

The front 120 of the bra 100 has two cups to hold the two breasts of a patient. The cups of the bra 100 are connected at the center of the front part of the bra by fasteners 125 for easy removal. These fasteners are at least two and preferably more vertical columns of metal and or plastic fasteners that engage one to the other so as to prevent the bra from disengaging. The at least two columns and preferably more columns are found one attached near one cup and the other attached near the other cup in a centralized vertical fashion in the general proximity of the central closing ends of the entire bra 100 at its front 120 section. To further support the breasts of a patient two bra straps 105 extend from the leftmost and rightmost top edge of the top front 120 of the spa bra. They extend upwards and then backwards towards a central region of identical material 110 that is further sewn into the backing 115 of the bra; this back 115 region forms one integral piece with the main material support of dual cups that goes from the back of the bra 115 to the sides and finally into the front region of the two cups 120. It should be understood that the connection between the ends of the two straps 105 and the central region 110 are optionally made up of two plastic and or metal connectors that facilitate the snugness of the overall bra fitting; this may be preferably be replaced with simple strap material as one unit instead of having the metal or plastic connectors therein. These metal or plastic connectors are formed from a two-looped piece that typically uses the two loops for threading of a strap end and for threading of a central region material end there through; the strap end is threaded into one entrance over a loop and back out and is adjustable; the central region material end is threaded into an opposite entrance over the other loop and back out where it is sewn to the central region and is typically non-adjustable. The central region 110 and backing 115 are optionally replaced with the simple connection of each strap to the line or curve of material that runs smoothly from each cup material to the other cup material (this would be considered the backing material in this implementation); thus, in this optional situation each strap connects independently at the front and at the back instead of at a central location as in the drawing.

Further, the spa bra of FIG. 1 shows two novel pocket enclosures 130 (made of similar or identical materials as the bra itself as disclosed previously) in silhouette that are designed to receive a gel pack of cold, hot or cold-hot material; each of these pockets 130 is located preferably underneath the cup material and is sewn into each cup at the bottom curve line of the pocket. Alternatively, the pocket enclosures 130 are located on the surface of the cup; however, it is preferable to have them as close as possible to the region of surgical trauma so that the position indicated in the drawings, underneath the cups, is the best suited to improving the health of the patient. It is very clear from the drawing, that the pockets 130 so disclosed, form a half-moon shape or kidney shape oriented somewhat horizontally and situated in the best way so as to associate the pockets with the region of surgical trauma for a muscle breast implantation surgical procedure. These procedures involve moving up from the bottom of the breast and as a consequence leave a painful path underneath the breast. Whilst a half-moon shape has been shown following the general traumatic region of a typical breast augmentation procedure, it should be understood that other patterns, pocket sizes, shapes are contemplated by the inventor. For example, depending upon the size of the trauma so inflicted on the patient, the pocket size is reducible or expandable to cover the decreased size or increased size. If the injury is laid out in a narrower or different curved structure or straightened, for example, the band of material is made narrower or curved differently or straightened depending upon the needs of the patient.

FIG. 2 illustrates an inside perspective view of an open spa bra 200 with the several fasteners detached from one another in accordance with the teachings of the preferred embodiment. With the several fasteners detached from one another and the flaps of the spa bra cups opened, the two inner under sides of the cups are visible. Here are shown in clear perspective the two half-moon pockets 210 reversed from the view shown in FIG. 1. Additionally, each pocket 210 shown is sewn into the underside of the material forming the two cups at the location where the bottom of the pocket 210 connects with the underside of the cup material. Each pocket also has an opening that permits the insertion of a gel pack 220 of hot, cold or hot and cold materials for therapeutic reasons. The upper lip of the pocket 210 closes snugly into the material of the gel pack if it fills the opening or alternatively the material of the underside of the cup material itself. The pocket 210 is made of such a flexible material as previously discussed that its reflexive action acts to close the compartment; in this fashion, the material itself serves as the closure for the opening formed by the pocket 210 against the backing of the cup.

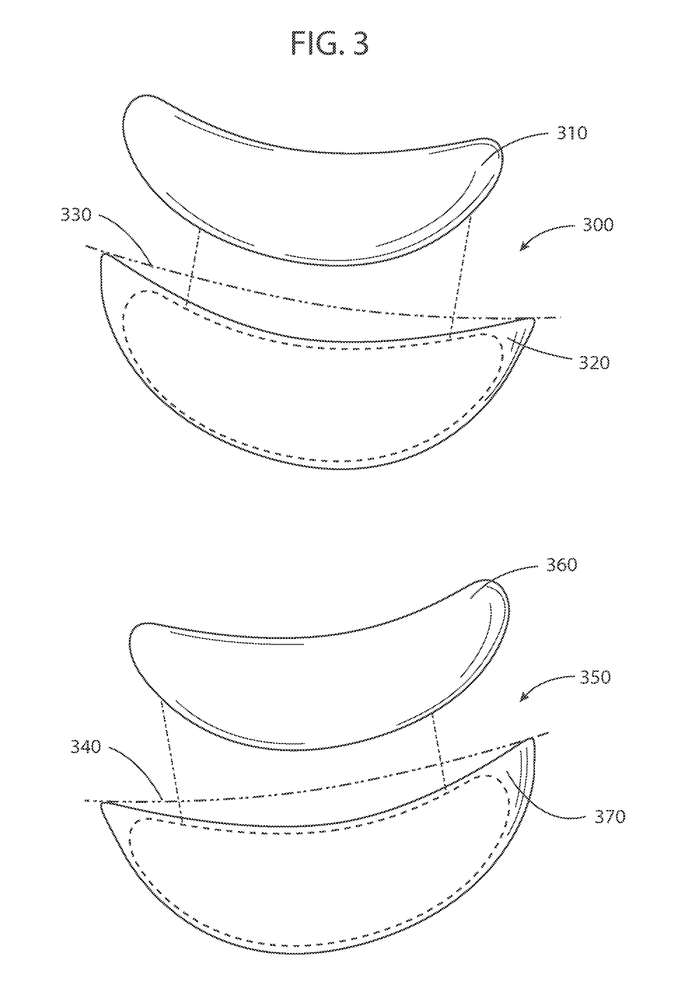

FIG. 3 illustrates the insertion of hot, cold or hot-cold gel packs into a pocket in accordance with the teachings of the preferred embodiment. FIG. 3 clearly shows the insertion 300 of a gel pack 310 into a rightmost pocket 320 of a bra (not shown). The bra pocket 320 mouth is opened by a user; then the gel pack 310 is carefully slid into place so as to avoid causing pain to the patient. It should be understood from the drawing that the line forming the back of the pocket 330 is not a separate piece of material like the top of the pocket 320. Rather, it is a section of the bra underside that is dotted to show how when closed, the mouth of the pocket closes into that shape. Similarly, FIG. 3 clearly shows the insertion 350 of a gel pack 360 into a leftmost pocket 370 of a bra (not shown). The bra pocket 370 mouth is opened by a user; then the gel pack 360 is carefully slid into place so as to avoid causing pain to the patient. It should be understood from the drawing that the line forming the back of the pocket 340 is not a separate piece of material like the top of the pocket 370. Rather, it is a section of the bra underside that is dotted to show how when closed, the mouth of the pocket closes into that shape.

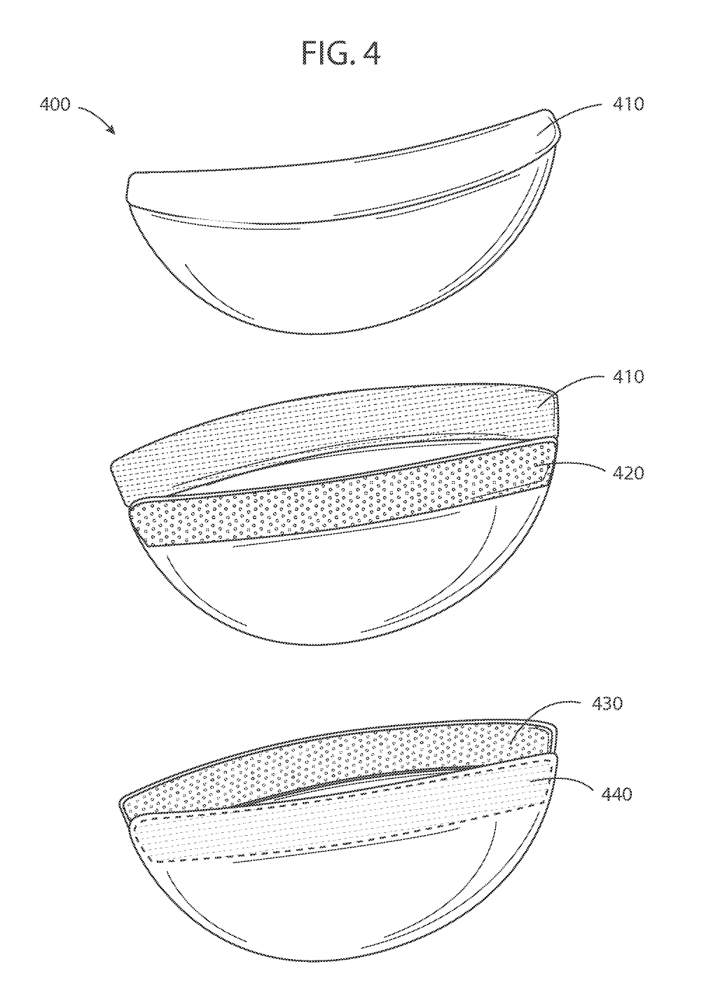

FIG. 4 illustrates an optional aspect of an alternative embodiment showing the closure of pockets utilizing small external and internal strips of corresponding hook and or loop Velcro (nylon and or polyester) strips of material in accordance with the teaching of the embodiment. Velcro is a material that has astounding attachment properties whereby a strip of fabric material (wool, cotton, linen etcetera) having hooks attaches into the hairy side of a material (wool, cotton, linen etcetera) having loops of corresponding material. FIG. 4 has three different drawings 400 showing various features of an optional nature. The figure shows a pocket having a closed flap 410 in the first drawing using Velcro type material; it 410 is sewn at one edge into the back side of the cup material in such a fashion so as to close over the opening of the pocket and come flip down upon the corresponding Velcro material 420. This same flap 410 is shown being opened in the second figure with hooks or loops Velcro material in the underside of the flap and the corresponding Velcro material of loops or hooks 420 is situated on the external lip of the pocket so as to make a opening and closure for the pocket. The final implementation illustrates a non-flap drawing where the Velcro strip of hooks or loops material is inserted into the internal lip of the pocket 440 (shown in silhouette). Then the corresponding Velcro strip of hooks or loops 430 is sewn into the underside of the two cups material directly facing the strip 440 so as to make a concealed closed connection for the pocket.

Final Considerations

As a result of having several rows of vertical fasteners, the bra is easy to adjust and can conform to many sizes that might be needed in the event of swelling as a result of the surgery. Thus, a patient who receives the bra will not have to buy a new one since increased swelling can be compensated for by the ability of the bra to adjust to size changes. Additionally, the bra maintains its sex appeal to women who have had such a surgery since its appearance is that of a typical bra with the additional benefit of having targeted pockets that close securely because of the material and the limited amount of material that makes it close readily for a snug fit; this provides for an expandable yet good fit. As taught herein, optional small Velcro flaps that are inserted at the top of the cup length also help ensure that the gel packs will not move out of the pockets. Additionally, whilst the gel packs have been shown as a single unit in a shape mimicking the general shape of the pockets herein they optionally may be any size and shape and used in combinations that work best for the closure of the overall system. Also, the pockets themselves are optionally situated on the outside surface of each cup rather than sewn to the inside surface as shown in the preferred embodiment. Another embodiment has only one cup with a pocket.

### CONCLUSION

Thus, the bra taught herein overcomes the prior art deficiencies by providing for a targeted placement of the gel packs close to the skin and directed to the specific location of the injury. The bra can be worn underneath ordinary clothing for everyday use as well as after surgery. Additionally, the small amount of material used, the specific shaping of pockets mimicking injuries and the variety of sizes the bra overcomes various prior art deficiencies. Amongst these are the motion problems, the spread of hot and or cold treatment to various locations that do not need such treatment and other problems found in the prior art. Further, a typical user will find that the use of this novel sports-type spa-bra not only helps to recover from a surgery but permits her to function in her ordinary lifestyle as well; this because the choice of materials and the simplicity of the implementation allows the bra to be utilized after the need for recovery has long since past. Its comfortable materials permit regular usage since it avoids the clumsy vest like materials found in the prior art.

The foregoing is considered illustrative only of the principles of the invention. Numerous changes may be made to the teachings herein without departing from the spirit and scope of the invention that should only be limited by the following claims.

I claim:

1. A therapeutic bra comprising:
a first cup connected to a first strap;
a second cup connected to a second strap;
a first strap further connected to a backing material;
a second strap further connected to the backing material; and
a first piece of material forming a first pocket with a portion of the first cup's material in specific physical symmetry with a location of a surgical injury on a first breast of a patient.

2. The therapeutic bra of claim 1, further comprising:
a second piece of material forming a second pocket with a portion of the second cup's material in specific physical symmetry with a location of a surgical injury on a second breast of a patient.

3. The therapeutic bra of claim 1, wherein the first piece of material forming a first pocket with a portion of the first cup's material in specific physical symmetry with a location of a surgical injury on a first breast of a patient is situated on the internal side of the first cup.

4. The therapeutic bra of claim 2, wherein the second piece of material forming a second pocket with a portion of the second cup's material in specific physical symmetry with a location of a surgical injury on a second breast of a patient is situated on the internal side of the second cup.

5. The therapeutic bra of claim 1, wherein the first piece of material forming a first pocket with a portion of the first cup's material in specific physical symmetry with a location of a surgical injury on a first breast of a patient is situated on the external side of the first cup.

6. The therapeutic bra of claim 2, wherein the second piece of material forming a second pocket with a portion of the second cup's material in specific physical symmetry with a location of a surgical injury on a second breast of a patient is situated on the external side of the second cup.

7. The therapeutic bra of claim 2, wherein the first and second cups' pockets are shaped as half-moons.

8. The therapeutic bra of claim 2, wherein the first and second cups' pockets are shaped as horizontal kidneys.

9. The therapeutic bra of claim 2, wherein the first and second cups' pockets are shaped according to shapes of injuries sustained on the respective breasts that they support.

10. The therapeutic bra of claim 1, wherein the first and second straps are connected to the backing material through a central material that is connected to the backing material.

11. The therapeutic bra of claim 1, further comprising:
at least two columns of vertical fasteners attached each nearby an opposite end of a cup so as to provide front bra closure when engaged one column of vertical fasteners to the other.

12. The therapeutic bra of claim 11, wherein there are more than two columns of vertical fasteners attached nearby ends of the cup supporting material so as to provide front bra closure when engaged one selected column to a selected column at another end.

13. The therapeutic bra of claim 1, further comprising:
a first therapy pack inserted into the first pocket to provide therapy.

14. The therapeutic bra of claim 2, further comprising:
a second therapy pack inserted into the second pocket to provide therapy.

15. The therapeutic bra of claim 3, further comprising:
a first therapy pack inserted into the first pocket to provide therapy.

16. The therapeutic bra of claim 4, further comprising:
a second therapy pack inserted into the second pocket to provide therapy.

17. The therapeutic bra of claim 5, further comprising:
a first therapy pack inserted into the first pocket to provide therapy.

18. The therapeutic bra of claim 6, further comprising:
a second therapy pack inserted into the second pocket to provide therapy.

19. A therapeutic bra comprising:
a first cup connected to a first strap;
a second cup connected to a second strap;
a first strap further connected to a backing material;
a second strap further connected to the backing material;
a first piece of material forming a first pocket with a portion of the first cup's material in specific physical symmetry with a location of a surgical injury on a first breast of a patient;
a second piece of material forming a second pocket with a portion of the second cup's material in specific physical symmetry with a location of a surgical injury on a second breast of a patient wherein the first and second cups' pockets are shaped according to shapes of injuries sustained on the respective breasts that they support.

20. A therapeutic bra comprising:
a first cup connected to a first strap;
a second cup connected to a second strap;
a first strap further connected to a backing material;
a second strap further connected to the backing material;
a first piece of material forming a first pocket with a portion of the first cup's material in specific physical symmetry with a location of a surgical injury on a first breast of a patient wherein the first cup's pocket is shaped according to the shape of an injury sustained on the breast that it supports.
